(19) United States
(12) Patent Application Publication
(10) Pub. No.: US 2006/0141026 A1
(43) Pub. Date: Jun. 29, 2006

(54) Imprinted paintball and apparatus and method for manufacturing same

(76) Inventor: Aldo Perrone, Brampton (CA)

Correspondence Address: HOFBAUER ASSOCIATES, SUITE 205 NORTH, 1455 LAKESHORE ROAD, BURLINGTON, ON, L7S 2J1, CA

(21) Appl. No.: 11/316,742

(22) Filed: Dec. 27, 2005

Related U.S. Application Data
(60) Provisional application No. 60/638,409, filed on Dec. 27, 2004.

(51) Int. Cl. A61K 9/48; A23G 1/22

(52) U.S. Cl. 424/451; 264/109; 425/117

(57) ABSTRACT

A gel capsule has embossed surface configurations that form, in relief, an imprinted design over the entire extent of its exterior surface. An apparatus and method for producing and/or manufacturing an imprinted gel ribbon that is useful in forming the gel capsule. The apparatus includes a spreader box and a rotatable casting drum. The spreader box holds a flowable gel, and has an opening through which the gel flows. The casting drum has an exterior cast surface that is provided with surface configurations in embossed relation thereon. In use, a receiving portion of the cast surface directly receives dispensed gel from the spreader box. The casting drum operatively rotates and drags the dispensed gel from the spreader box opening to solidify and form the gel ribbon, with the surface configurations forming, in relief, the imprinted design in one side of the ribbon.

### FIELD OF THE INVENTION

The present invention relates to the field of gel capsules and associated methods of manufacture, and, more specifically, to an imprinted gel capsule, such as might be useful, for example, as a paintball, and-to apparatus and methods for manufacturing same.

### BACKGROUND OF THE INVENTION

Capsules are solid dosage forms in which fill materials are enclosed in a gel wall. The fill materials may include paintball fill materials and therapeutic agents, and at least in the case of the latter, the gel wall of prior art capsules has typically been formed as a soluble gelatin wall. The wall of a gelatin capsule can be made of either soft or hard gelatin. In the prior art, the gelatin may in some cases have been plasticized by the addition of additives such as glycerin, sorbitol, and/or similar types of polyols. Soft gelatin capsules have offered a convenient dosage form for the administration of drugs, nutrients, vitamins, foodstuff and cosmetics. Commercially available gelatin capsules have been provided in a variety of sizes and shapes, e.g., round, oval, oblong, tubular, and suppository-shaped.

In the prior art, gel capsules have typically been formed by joining two identical halves to form a shell encapsulating the fill material. Generally, prior art soft gelatin capsules have been prepared by the rotary-die process. In this process, soft gelatin capsules have been made by continuously casting two separate ribbons of molten or flowable gelatin into two separate rotating dies of an encapsulation machine to produce soft, elastic gelatin capsules. Four prior art patent references, namely, (i) U.S. Pat. No. 5,672,300 issued to Schurig et al. on Sep. 30, 1997 for a Method for Striping or Marbleizing Capsules, (ii) U.S. Pat. No. 6,022,499 issued to Schurig et al. on Feb. 8, 2000 for a Method for Striping or Marbleizing Capsules, (iii) U.S. Pat. No. 6,569,363 issued to Schurig et al. on May 27, 2003 for Apparatus and Methods for Forming Patterned Soft Gelatin Capsules, and (iv) U.S. patent application Ser. No. 10/373,158 (Schurig et al.) for a Apparatus and Methods for Forming Patterned Soft Gelatin Capsules (published on Jul. 10, 2003 under U.S. Patent Publication No. 2003/0127760), provide a description of well known soft gelatin capsule art. The rotary die processes described in these references are incorporated herein by reference as being descriptive of the rotary die process to which the present invention is related.

While teachings of the foregoing references are limited to methods and apparatus for making capsules having gelatin walls that include two or more visually distinct gelatins, other prior art references have gone further to teach limited methods of imprinting identifying marks into the exterior surfaces of soft gel capsule shells. For example, U.S. Pat. No. 2,688,775 (Scherer) shows a method for applying a brand to the exterior surface of a nearly finished gelatin capsule. Similarly, U.S. Pat. No. 2,703,047 (Scherer) discloses a system of branding already filled capsules. In other prior art references, including, for example, U.S. Pat. No. 3,124,840 (Taylor) and German Patent No. 3,181,071, a substantially finished gelatin strip or ribbon is imprinted with a simple design just prior to manufacturing capsules therefrom. U.S. Pat. No. 2,596,176 (Scherer) teaches a relatively primitive method as well as an apparatus for striping gelatin capsules, wherein the gelatin capsule is grasped at its ends and parts thereof are abraded, with a colored material thereafter being applied thereto.

The prior apparatus and methods for making soft gelatin capsules have failed to provide a convenient, easy to use, reproducible apparatus and method for producing imprinted gel capsules, such as, for example, soft gelatin capsules and/or paintball capsules. Moreover, the prior methods, such as they are, have been unsatisfactory, laborious, and/or have lacked reproducibility. There is a need in the art for an apparatus and method to solve these and other problems.

At this stage, it is worthwhile to specifically discuss some of the problems with the prior art related to paintballs and apparatus and methods for manufacturing same. Heretofore, paintballs have a suffered from number of problems, including the following: prior art paintballs often (a) are not round; (b) have had poor or unreliable performance characteristics; (c) have exhibited unpredictable flight trajectories and inconsistent accuracies (depending, inter alia, on the shape and/or roundness of prior art paintballs, they have tended to travel towards the direction of the heavier portion of the ball); (d) have tended to create a good deal of friction in the barrel of the paintball gun; (e) have tended to create a significant amount of air turbulence; (f) have not been optimized to be fired a great distance; and/or (g) have had generally poor esthetics. Additionally, it has not previously been possible to imprint complex textures, patterns, impressions, and/or logos, in embossed relation, on the skin of the paintball capsules. Likewise, it has not heretofore been possible to imprint complex patterns, textures, impressions, and/or logos, in embossed relation, on the gelatin skin of bath bead shells or capsules for carrying therapeutic agents and the like.

Previous manufacturing methods aimed at imprinting paintballs have been subject to a number of problems, as follows: (a) they have only been capable of imprinting relatively simple patterns on the exterior surfaces of paintballs; (b) any imprinted designs have heretofore been confined to limited portions of the capsule's shell, rather than substantially covering an entire exterior surface thereof; (c) imprinted designs may have consisted solely of lines that have been imprinted on a paintball ribbon in substantially parallel relation with a direction of the ribbon's travel over and/or away from any casting drum; and (d) they may have involved the use of a secondary roller substantially engaging the casting drum, with consequent increased risk of damaging the solidifying paintball ribbon and/or requiring substantially constant supervision. All such previous methods have been substantially unreliable and/or have required substantially constant human supervision at a spreader box to ensure that a liquid gel might be properly spread onto a casting drum to form a paintball ribbon, before ultimately becoming the surface of a paintball's gelatin skin. None of the prior art has disclosed the direct use of surface configurations provided on the casting drum to imprint impressions, logos, patterns, textures, etc. onto a gelatin skin of a paintball or capsule.

It is an object of the invention to obviate or mitigate at least one of the above mentioned disadvantages and/or problems associated with the prior art.

Accordingly, and without limitation, it is an object of one aspect of the invention to provide imprinted paintballs that may be beneficially used, inter alia, in association with recreational, tournament, and survival variations of a paintball game or pastime. Preferably, but not necessarily, such imprinted paintballs would afford (i) improved performance characteristics when used in association with a paintball game or pastime, and/or (ii) improved flight trajectory and accuracy when fired from a paintball marker or gun. Further, in some cases, such imprinted paintballs would preferably, but not necessarily, (iii) create less friction in the barrel of a paintball gun so that it might be fired a greater distance, and/or (iv) exhibit reduced turbulence when fired from a paintball gun. Similarly, it is an object of some aspects of the invention to provide an imprinted paintball that might be used as an imprinted bath bead shell, and/or as an imprinted gelatin capsule for carrying a therapeutic agent, or the like.

It should also be appreciated that, among other things, is an object of some aspects of the invention to provide a method of manufacturing an imprinted gel capsule that does not require substantially constant supervision at a spreader box, whereat a liquid gel might be spread onto a casting drum.

### SUMMARY OF THE INVENTION

In accordance with the present invention there is disclosed an apparatus for producing an imprinted gel ribbon useful for forming imprinted gel capsules. The apparatus includes a spreader box and a rotatable casting drum. The spreader box is adapted to hold a flowable gel mass. The spreader box defines a spreader box opening that is adapted to enable operative flow of the gel mass therethrough. The rotatable casting drum has an exterior cast surface. The cast surface is provided with surface configurations in embossed relation thereon. A receiving portion of the cast surface is in substantial registration with the spreader box opening so as to be adapted to directly receive a dispensed portion of the gel mass therefrom. The casting drum operatively rotates with one or more rotating speeds and is adapted to operatively drag the dispensed portion of the gel mass from the spreader box opening to solidify and form the imprinted gel ribbon. The surface configurations on the cast surface of the casting drum are adapted to form, in relief, an imprinted design in one side of the imprinted gel ribbon.

In some cases, the apparatus may also include a cooling means for operatively cooling the cast surface of the casting drum, such as, for example, a forced air ventilation system.

In some cases, the surface configurations on the cast surface of the casting drum may be shaped to form one or more imprinted designs selected from the group consisting of an embossed logo, an embossed pattern, and an embossed texture, such as, for example, an embossed dimpled texture.

In some cases of the apparatus according to the invention, the spreader box opening and/or the rotating speed of the casting drum may be adapted to operatively form the imprinted gel ribbon as a substantially continuous ribbon and/or having a thickness substantially in the order of about 0.035 inches before any die rolling. Similarly, in some cases of the apparatus according to the invention, a width of the casting drum and/or the spreader box opening may be sized and adapted to operatively form the imprinted gel ribbon having a width substantially in the order of about five inches.

In some cases, according to the invention, the apparatus may be further adapted to form imprinted gel capsules. In such cases, the apparatus may also include transport means and dispensing means, among other things. The transport means is preferably adapted to provide a pair of the imprinted gel ribbons towards, and/or between, a pair of rotary dies. The dispensing means is preferably provided in order to dispense, preferably, but not necessarily, under pressure, a fill material to an injection wedge located between the rotary dies.

In accordance with the present invention there is also disclosed a method for producing an imprinted gel ribbon that is useful for forming imprinted gel capsules. According to this method, a casting drum is provided having an exterior cast surface, with surface configurations formed in embossed relation thereon. Thereafter, a dispensed portion of a gel mass contained in a spreader box is flowed, through an opening in the spreader box opening, directly onto a receiving portion of the exterior cast surface of the casting drum. The casting drum rotates so as to drag and solidify the dispensed portion of the gel mass, such that the imprinted gel ribbon is formed, with the surface configurations on the cast surface of the casting drum forming, in relief, an imprinted design in one side of the imprinted gel ribbon.

In some cases, according to the invention, the method also includes a step of separating the imprinted gel ribbon from the casting drum as a substantially continuous ribbon.

In some cases of the method according to the invention, the imprinted gel ribbon that is so produced may used to form an imprinted gel capsule by the rotary die method.

In some such cases, a pair of imprinted gel ribbons may formed by the above method. Thereafter, the ribbons may be fed between a pair of rotary dies containing matching die pockets, whereby a single web of imprinted gel material is formed having encapsulated pocket portions defined therewithin. The encapsulated pocket portions are filled with a fill material.

In some cases of the method according to the invention, the imprinted design may be formed, in relief, in both sides of the single web of imprinted gel material.

In some cases of the method according to the invention, an encapsulating wall of the web, adjacent to the encapsulated pocket portions, may be substantially in the order of about 0.008 inches in thickness.

In some cases of the method according to the invention, the encapsulated pocket portions may be separated from the web of imprinted gel material so as to form the imprinted gel capsules.

In accordance with the present invention there is also advantageously disclosed a novel and inventive imprinted and filled gel capsule that may be produced using the method and/or apparatus described hereinabove. The gel capsule includes a capsule portion defining an interior encapsulated pocket therewithin, and a fill material that is contained within the encapsulated pocket of the capsule portion. The capsule portion is formed from a solidified gel material, and has an exterior surface that is provided, in embossed relation, with surface configurations. Advantageously, the surface configurations form, in relief, an imprinted design over substantially the entire extent of the exterior surface of the capsule portion.

In some cases of the gel capsule according to the invention, the surface configurations may be shaped to form one or more imprinted designs selected from the group consisting of an embossed logo, an embossed pattern, and an embossed texture, such as, for example, an embossed dimpled texture.

In some cases of the gel capsule according to the invention, the capsule portion has a capsule wall that may be substantially in the order of about 0.008 inches in thickness.

In some cases of the gel capsule according to the invention, the gel material may include gelatin, glycerin, sorbitol, water, titanium, and/or one or more dyes. Similarly, in some cases of the gel capsule according to the invention, the gel material may include a synthetic gel material, such as, for example, starches and/or other polymers.

In some cases of the gel capsule according to the invention, the fill material may include one or more fill materials selected from the group consisting of paintball fill materials, bath bead fill materials, pharmaceutical fill materials, and/or nutraceutical fill materials.

As will be appreciated from the foregoing, and as aforesaid, it is an object of this invention to obviate or mitigate at least one of the above mentioned disadvantages of the prior art.

Other advantages, features and characteristics of the present invention, as well as methods of operation and functions of the related elements of the structure, and the combination of parts and economies of manufacture, will become more apparent upon consideration of the following detailed description and the appended claims with reference to the accompanying drawings, the latter of which is briefly described hereinbelow.

### BRIEF DESCRIPTION OF THE DRAWINGS

The novel features which are believed to be characteristic of the according to the present invention, as to its structure, organization, use and method of operation, together with further objectives and advantages thereof, will be better understood from the following drawings in which a presently preferred embodiment of the invention will now be illustrated by way of example. It is expressly understood, however, that the drawings are for the purpose of illustration and description only, and are not intended as a definition of the limits of the invention. In the accompanying drawings:

FIG. 1 is a schematic drawing of an apparatus and method for manufacturing imprinted gel ribbons and capsules according to the invention;

FIG. 2 is a schematic drawing of the apparatus and method for manufacturing imprinted gel ribbons shown in FIG. 1;

FIG. 3 is a sectional view of a spreader box, casting drum, and imprinted gel ribbon shown in FIG. 2;

FIG. 4 is a top plan view of a portion of the imprinted gel ribbon of FIG. 3, showing an imprinted surface thereof;

FIG. 5 is a sectional view of an injection wedge, rotary dies, and web of imprinted gel material shown in FIG. 1;

FIG. 6A is a front elevational view of a gel capsule according to the invention, showing a dimpled texture imprinted in an exterior surface thereof;

FIG. 6B is a sectional view of the imprinted gel capsule of FIG. 6A along sight line 6B-6B; and

FIG. 7 is a front elevational view of a gel capsule according to the invention, showing a pattern of logos imprinted in the exterior surface thereof.

### DETAILED DESCRIPTION OF A PREFERRED EMBODIMENT

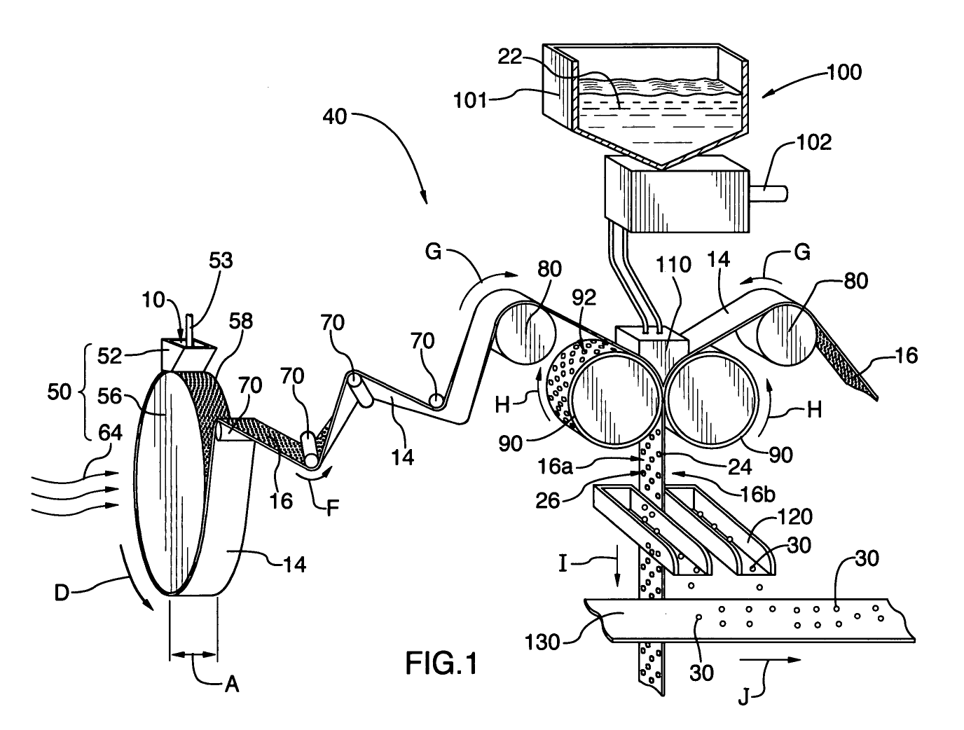

Referring now to FIG. 1 of the drawings, there is shown a schematic drawing of a rotary die apparatus 40 for forming imprinted gel capsules 30 according to the invention. The rotary die apparatus 40 includes a ribbon-making apparatus 50 for producing an imprinted gel ribbon 14 that is useful for forming the imprinted gel capsules 30. The ribbon-making apparatus 50 includes a spreader box 52 and a rotatable casting drum 56.

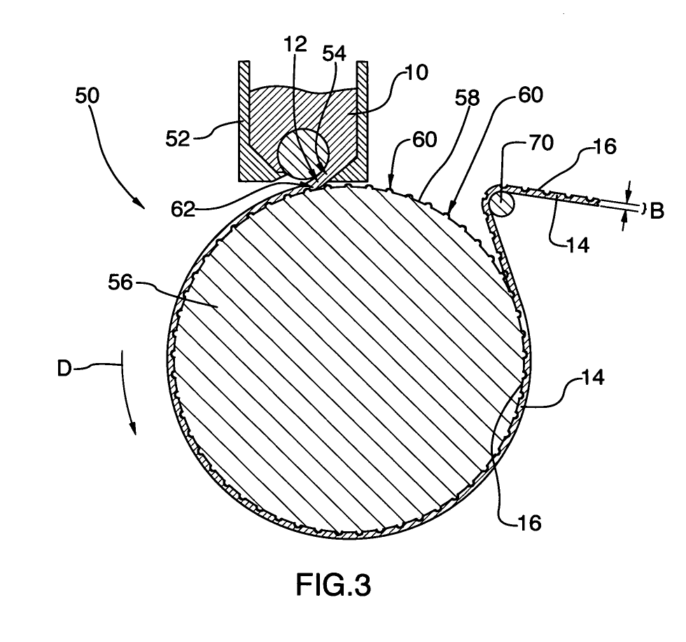

As best seen in FIG. 3, the spreader box 52 is adapted to hold a flowable gel mass 10. As shown in FIG. 3, the spreader box 52 defines a spreader box opening 54 that is adapted to enable operative flow of the gel mass 10 therethrough.

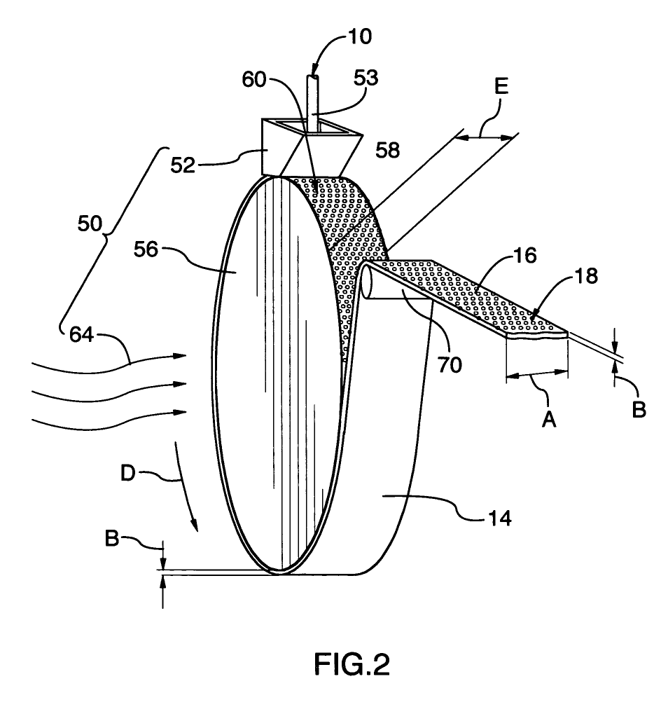

As best seen in FIG. 2, the casting drum 56 has an exterior cast surface 58 that is provided with surface configurations 60 in embossed relation thereon. The surface configurations 60 on the cast surface 58 of the casting drum 56 are shaped to form at least one imprinted design 18 selected from the group comprising, but not limited to, an embossed logo, an embossed pattern, and an embossed texture, such as, for example, an embossed dimpled texture (as shown in FIG. 2). One skilled in the art will recognize, however, that the imprinted design may be of any other suitable or desired geometry.

As shown in FIG. 3, a receiving portion 62 of the cast surface 58 is in substantial registration with the spreader box opening 54 so as to directly receive, in operation, a dispensed portion 12 of the gel mass 10 therefrom.

In use, and as best appreciated from FIG. 3, the casting drum 56 operatively rotates (in the direction generally indicated by arrow “D” shown in FIGS. 1 to 3) and drags the dispensed portion 12 of the gel mass 10 from the spreader box opening 54 to solidify and form the imprinted gel ribbon 14. The surface configurations 60 on the cast surface 58 of the casting drum 56 form, in relief, an imprinted design 18 in one side 16 of the imprinted gel ribbon 14.

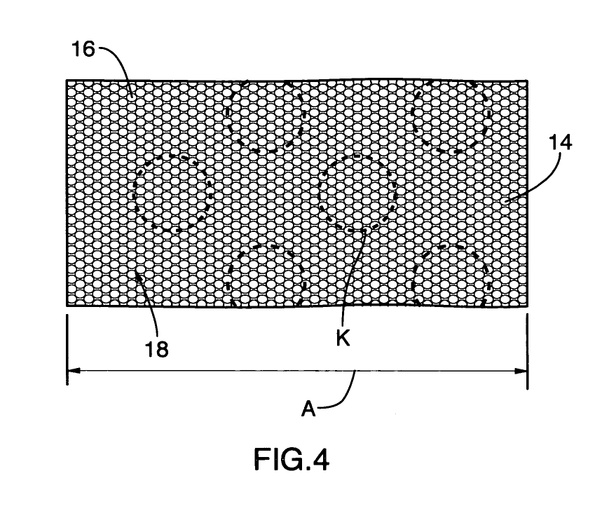

A portion of an exemplary ribbon 14 is shown in FIG. 4. In the example shown, one side 16 of the ribbon is provided with the imprinted design 18. It should be appreciated that, advantageously and according to the invention, the imprinted gel ribbon 14 may be useful in association with production and/or manufacture of gel capsules 30. The outline of the gel capsules 30 is generally depicted in an exemplary position relative to the imprinted gel ribbon 14 by way of phantom outline “K” which is shown in FIG. 4.

As generally shown in FIGS. 1 and 2, the ribbon making apparatus 50 may, in some cases, further include a cooling means 64 (alternately hereinafter referred to as a forced air ventilation system 64) that operatively cools the cast surface 58 of the casting drum 56. Instead of, and/or in addition to, the forced air ventilation system 64, the cooling means may take the form of a refrigeration system (not shown), and/or any variety of other systems that are suitable to thermodynamically extract and/or recover heat from the casting drum 56.

Preferably, but not necessarily, both the spreader box opening 54 and the rotating speed of the casting drum 56 are adapted to ensure that the imprinted gel ribbon 14 is formed as a substantially continuous strip of material. Additionally, the respective widths of both the casting drum 56 (as indicated generally by dimension “E” shown in FIG. 2) and the spreader box opening 54 are preferably, but not necessarily, sized to operatively provide the imprinted gel ribbon 14 with a width (as indicated generally by dimension “A” shown in FIGS. 1 and 2) of about five inches. One skilled in the art will recognize that the gel ribbon 14 may, alternately, be provided with any other suitable or desired width “A”, such as, for example, 3 inches, 6 inches, 24 inches, or indeed with any suitable or desired, lesser or greater, width “A”.

Preferably, but not necessarily, both the spreader box opening 54 and the rotating speed of the casting drum 56 are further adapted to ensure that the imprinted gel ribbon 14 is formed with a thickness (as indicated generally by dimension “B” shown in FIG. 2) of about 0.035 inches before any die rolling (which die rolling will be described in further detail hereinbelow). One skilled in the art will recognize that the gel ribbon 14 may, alternately, be provided with any other suitable or desired thickness “B” before die rolling, such as, for example, 0.010 inches, 0.050 inches, or indeed with any suitable or desired, lesser or greater, thickness “B” before die rolling. As previously mentioned, the die rolling process is described in further detail hereinbelow.

As best seen in FIG. 1, the rotary die apparatus 40 may preferably, but not necessarily, also include oil rolling means 70, transport means 80, dispensing means 100, and/or chute means 120.

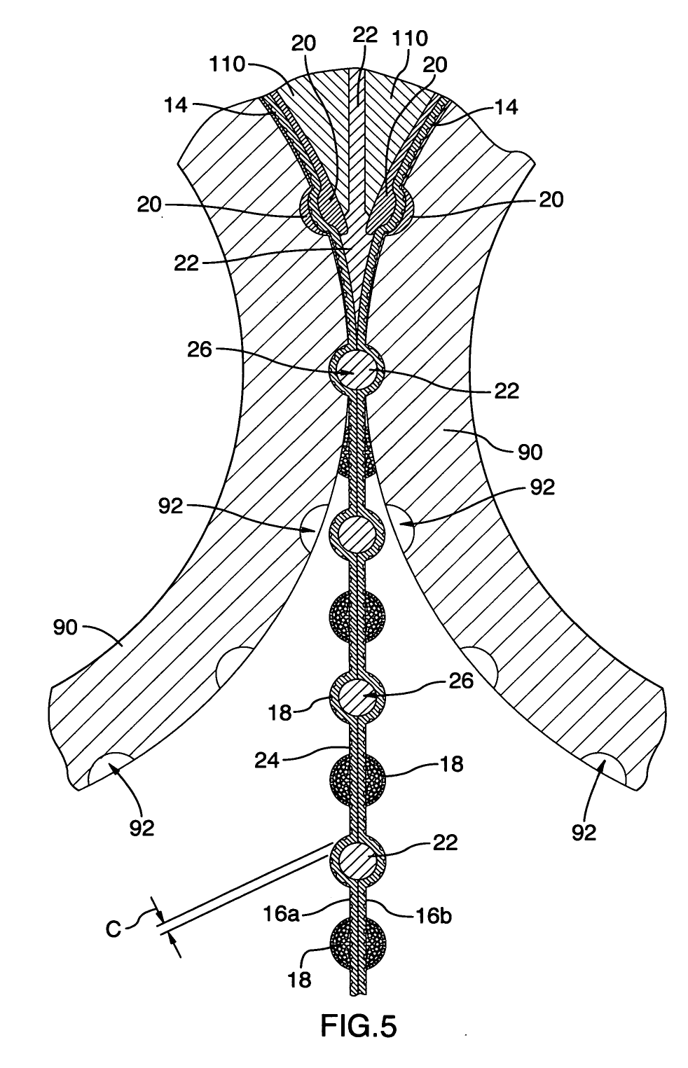

In use, the oil rolling means 70, shown in FIG. 1 generally as a series of oil rollers, preferably ensures that the imprinted gel ribbon 14 is covered with an oil layer 20 (best seen in FIG. 5) as it moves away from the casting drum 56 (in the general direction of arrow “F” shown in FIG. 1).

The transport means 80 is shown in FIG. 1 generally as a pair of feeder rollers. The transport means 80 provides a pair of the imprinted (and preferably oil covered) gel ribbons 14 towards (in the general direction of arrow “G” shown in FIG. 1), and/or between, a pair of rotary dies 90. There, the two gel ribbons 14 substantially merge (as will be described in further detail hereinbelow).

As shown in FIG. 1, the dispensing means 100 may include a reservoir 101 containing a capsule fill material 22 and a fill pump 102 that may dispense the fill material 22, preferably under pressure, to an injection wedge 110 that is located between the rotary dies 90.

As best seen in FIG. 5, the injection wedge 110 may preferably, but not necessarily, dispense the fill material 22 into a gap between the merging pair of imprinted gel ribbons 14.

From the pair of imprinted gel ribbons 14, the rotary dies 90, rotating generally towards one another (as indicated generally by arrows “H” shown in FIG. 1), form a single web 24 of imprinted gel material. As best seen in FIG. 5, the web 24 preferably, but not necessarily, has the imprinted design 18 formed, in relief, in both sides 16a, 16b thereof.

As best seen in FIG. 5, the rotary dies 90 define matching die pockets 92, such that the injection wedge 110 may preferably dispense the fill material 22, preferably under pressure, into encapsulated pocket portions 26 that are thereby formed and defined within the web 24.

As shown in FIG. 1, the web 24 travels away from the rotary dies 90, 90 (in the general direction of arrow “I”). The encapsulated pocket portions 26 are subsequently separated from the web 24, thus becoming the discrete imprinted gel capsules 30, which are then received by the chute means 120.

A conveyer 130 may thereafter carry the imprinted gel capsules 30 (in a direction indicated generally by arrow “J” shown in FIG. 1) to a tumble dryer (not shown). The conveyor 130 need not necessarily be present and, in fact, the chute means 120 may feed directly into the tumble dryer and/or into some other container. In those cases where it is present, the tumble dryer (not shown) may be used to harden the gel capsules 30 for their ultimate intended use, e.g., as paintballs.

A method for producing the imprinted gel capsules 30, including a method for producing the imprinted gel ribbons 14 that may be useful in forming the gel capsules 30, according to the present invention will now be briefly described with reference to the figures. It will be appreciated by one skilled in the art that the method outlined hereinbelow is but one such method that falls within the scope of the invention as circumscribed by the appended claims. In the following description, the same reference numerals have been used to indicate various components, surfaces, materials, relations, directions, and configurations which are common to both the method and the rotary die apparatus 40 (described above) of the present invention. It should, however, be appreciated that, although some of the components, surfaces, materials, relations, directions, and configurations of the rotary die apparatus 40 are not specifically referenced in the following description of the method, they may be used, and/or adapted for use, in association therewith.

Now, therefore, according to the method, the casting drum 56 is provided with its exterior cast surface 58. The cast surface 58 has the surface configurations 60 formed in embossed relation thereon.

As best seen in FIGS. 1 and 2, a tube 53 may, in some cases, provide the gel mass 10 to spreader box 52. In this way, a constant supply of the gel mass 10 may be easily maintained in the spreader box 52.

Next, the dispensed portion 12 of the gel mass 10 from the spreader box 52 is flowed, through the spreader box opening 54, directly onto the receiving portion 62 of the cast surface 58. In this manner, the gel mass 10 in the spreader box 52 may be spread onto the cast surface 58 of the casting drum 56. The gel mass 10 may advantageously comprise, for example and without limitation, a gelatin mass.

The casting drum 56 is rotated in the direction “D” so as to drag and solidify the dispensed portion 12 of the gel mass 10, and such as to form the imprinted gel ribbon 14. In some cases of the method, the cast surface 58 of the casting drum 56 may be positively cooled as it rotates and drags the dispensed portion 12 of the gel mass 10.

In the aforesaid manner, and as best seen in FIG. 2, the surface configurations 60 on the cast surface 58 of the casting drum 56 form, in relief, the imprinted design 18 in one side 16 of the imprinted gel ribbon 14. It will be appreciated that the design 18 imprinted in the ribbon 14 is a reverse image of that on the casting drum 56.

According to the method of the invention, the gel ribbon 14 may preferably, but not necessarily, be about 0.035 inches in thickness “B” before any die rolling. As previously mentioned, one skilled in the art will recognize that the gel ribbon may, alternately, be provided with any other suitable or desired, lesser or greater, thickness “B” before die rolling.

In some cases of the method according to the invention, the imprinted gel ribbon 14 is preferably, but not necessarily, separated from the casting drum 56 as a substantially continuous ribbon.

In some cases of the method, the imprinted gel ribbon 14 may be used to form the imprinted gel capsule 30 by the rotary die method.

In some such cases, a pair of the imprinted gel ribbons 14 may be formed, each substantially according to the steps described hereinabove. Alternately, for example and without limitation, only one of the pair of imprinted gel ribbons 14 may be provided with the imprinted design 18, and/or the two may be provided with different imprinted designs (not shown), thereon.

In some cases of the rotary die method according to the invention, the imprinted gel ribbons 14 may then be covered with the oil layer 20 (best seen in FIG. 5).

In the rotary die method, the ribbons 14 are fed between the pair of rotary dies 90. As best seen in FIG. 5, the oil layer 20 may take the form of an oil bead that is situated between the ribbon 14 and the rotary die 90 and between the ribbon 14 and the injection wedge 110. That is, the beads of the oil layer 20 may be substantially juxtaposed between each ribbon 14 and a corresponding rotary die 90, and between each ribbon 14 and the injection wedge 110. In this manner, the ribbons 14 may preferably, but not necessarily, be kept from becoming stuck to the injection wedge 110.

The rotary dies 90 contain the matching die pockets 92, such that the rotary dies 90 form the single web 24 of imprinted gel material from the pair of gel ribbons 14, with the encapsulated pocket portions 26 being defined therewithin.

The encapsulated pocket portions 26 (alternately hereinafter referred to as interior encapsulated pockets 26) are filled with the fill material 22. The fill material 22 may, actually, consist of one or more fill materials 22 that are selected from the group that comprises, without limitation, paintball fill materials, bath bead fill materials, pharmaceutical fill materials, and nutraceutical fill materials.

From the foregoing, and as shown in FIGS. 1 and 5, it will be appreciated that the pair of ribbons 14, 14 meet one another and substantially merge between the rotary dies 90, 90. There, they are formed into the web 24, with the fill pump 102 feeding the fill material 22, under pressure, through the injection wedge 110 to form a plurality of the pocket portions 26 in the web 24.

Preferably, but not necessarily, an encapsulating wall 28 (alternately hereinafter referred to as a capsule wall 28) of the web 24, adjacent to the encapsulated pocket portions 26, may be about 0.008 inches in thickness (as indicated generally by dimension line “C” in FIG. 5). One skilled in the art will recognize that the encapsulating wall 28 may, alternately, be provided with any other suitable or desired, lesser or greater, thickness “C”.

In some cases, and as mentioned hereinabove, the imprinted design 18 may preferably be formed, in relief, in both sides 16a, 16b of the single web 24 of imprinted gel material.

In some cases of the rotary die method according to the invention, the encapsulated pocket portions 26 may then preferably be separated from the web 24 of imprinted gel material so as to form the imprinted gel capsules 30.

Imprinted (and filled) gel capsules 30, according to the present invention, will now be briefly described with reference to the figures. It will be appreciated by one skilled in the art that the capsules 30 described hereinbelow represent but a few such capsules that fall within the scope of the invention as circumscribed by the appended claims. In the following description, the same reference numerals have been used to indicate various components, surfaces, materials, relations, directions, and configurations which are common both to the capsules 30 and to the method and apparatus 40 (described above) of the present invention. It should, however, be appreciated that, although some of the components, surfaces, materials, relations, directions, and configurations of the apparatus 40 and method are not specifically referenced in the following description of the capsules 30, they may be used, and/or adapted for use, in order to produce and/or manufacture same.

Now, therefore, each imprinted and filled gel capsule 30 according to the invention may preferably include a capsule portion 32 that defines the interior encapsulated pocket 26, as well as a fill material 22 that is contained within the encapsulated pocket 26. As aforesaid, the fill material 22 may, actually, consist of one or more fill materials 22 that are selected from the group that comprises, without limitation, paintball fill materials, bath bead fill materials, pharmaceutical fill materials, and nutraceutical fill materials.

In the capsules 30 according to the invention, the capsule portion 32 is formed from a solidified gel material. The gel material may advantageously comprise, for example and without limitation, gelatin. In some such cases, the gel material may preferably, but not necessarily, also include one or more of the following constituents, among others: glycerin, sorbitol, water, titanium, and/or one or more dyes.

In addition to the above, and/or alternately, the gel material may advantageously comprise, for example and without limitation, a synthetic gel material. In some such cases, the synthetic gel material may preferably, but not necessarily, include one or more starches and/or other polymers.

Advantageously, in the capsules 30 according to the invention, an exterior surface 34 of the capsule portion 32 is provided, in embossed relation, with surface configurations 36 that form, in relief, the imprinted design 18 over substantially the entire extent thereof. It bears mentioning, once again, that the prior art has not heretofore afforded an ability to imprint a design over the entire surface of a capsule.

As aforesaid, the surface configurations 36 may preferably be shaped to form at least one imprinted design 18 selected from the group that comprises, but is not limited to, an embossed logo, an embossed pattern, and an embossed texture.

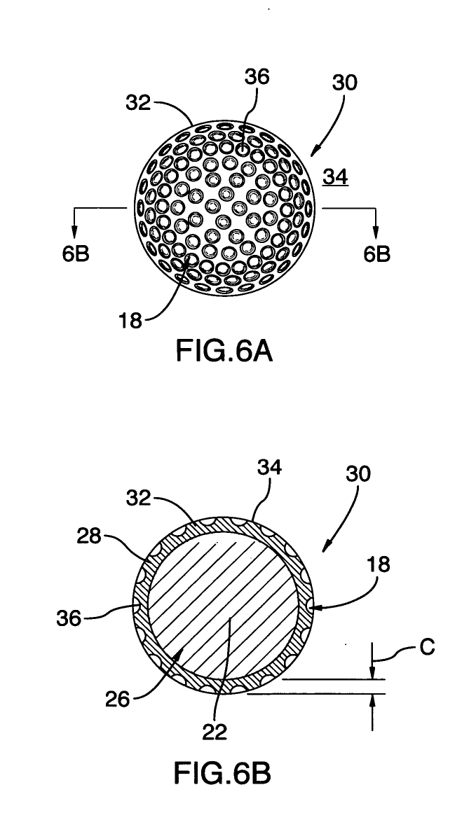

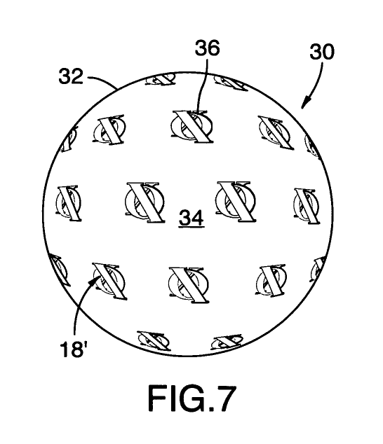

Advantageously, and as best seen in FIG. 6A, the imprinted design may be an embossed dimpled texture. One skilled in the art will recognize that the imprinted design may likewise be of any other suitable geometry, such as, for example, a pattern of embossed logos 18′ (as shown in FIG. 7).

Preferably, but not necessarily, the capsule wall 28 of the capsule portion 32 may be about 0.008 inches in thickness “C” (as shown in FIG. 6A). One skilled in the art will recognize that the encapsulating wall 28 may, alternately, be provided with any other suitable or desired, lesser or greater, thickness “C”.

From the foregoing, it should be appreciated that, depending in part on any specific fill material that may be selected, the imprinted gel capsules 30 may be suitable for use as paintballs, bath beads, and/or therapeutic capsules, among other uses.

In use as paintballs, the imprinted gel capsules 30 according to the invention may exhibit substantially improved performance characteristics in comparison with prior art paintball capsules, including one or more of the following: (a) improved flight trajectories and accuracies (having a tendency to travel much straighter); (b) the creation of less friction in the barrel of a paintball gun; (c) the ability to be fired over a greater distance (possibly in the range of an extra 50′ or more); and/or (d) a reduced turbulence when fired from a paintball gun. It is generally thought that the dimpled texture of the imprinted design 18 on the capsule 30 may provide improved flight characteristics and/or other advantages.

Generally, the imprinted capsules 30 according to the invention may have a more rounded shape, and/or a generally more esthetically pleasing shape, that may be advantageous in a wide variety of applications and/or intended uses, whether as paintballs, bath beads, therapeutic gel capsules, and/or the like.

Additionally, the method described hereinabove may advantageously enable and/or facilitate the creation of imprinted textures, patterns, impressions, and/or logos on the surface of gel ribbons 14 and/or capsules 30, without requiring substantially constant human supervision at the spreader box 52 to ensure that the gel mass 10 is properly spread onto the casting drum 56.

Of course, other modifications and alterations may be used in the design and manufacture of other embodiments according to the present invention without departing from the spirit and scope of the invention, which is limited only by the accompanying claims.

I claim:

1. An apparatus for producing an imprinted gel ribbon useful for forming imprinted gel capsules, said apparatus comprising:
a) a spreader box adapted to hold a flowable gel mass, with said spreader box defining a spreader box opening that is adapted to enable operative flow of the gel mass therethrough;
b) a rotatable casting drum having an exterior cast surface, with said cast surface being provided with surface configurations in embossed relation thereon, and with a receiving portion of said cast surface being in substantial registration with said spreader box opening so as to be adapted to directly receive a dispensed portion of the gel mass therefrom;
wherein said casting drum operatively rotates with at least one rotating speed and is adapted to operatively drag the dispensed portion of the gel mass from said spreader box opening to solidify and form said imprinted gel ribbon, with said surface configurations on said cast surface of said casting drum being adapted to form, in relief, an imprinted design in one side of said imprinted gel ribbon.

2. An apparatus according to claim 1, further comprising cooling means for operatively cooling said cast surface of said casting drum.

3. An apparatus according to claim 2, wherein said cooling means comprises a forced air ventilation system.

4. An apparatus according to claim 1, wherein said surface configurations on said cast surface of said casting drum are shaped to form at least one imprinted design selected from the group consisting of an embossed pattern, an embossed texture, and an embossed logo.

5. An apparatus according to claim 4, wherein said surface configurations on said cast surface of said casting drum are shaped to form an embossed dimpled texture.

6. An apparatus according to claim 1, wherein at least one of said spreader box opening and said rotating speed is adapted to operatively form said imprinted gel ribbon as a substantially continuous ribbon.

7. An apparatus according to claim 1, wherein at least one of a width of said casting drum and said spreader box opening is sized and adapted to operatively form said imprinted gel ribbon having a width substantially in the order of about five inches.

8. An apparatus according to claim 1, wherein at least one of said spreader box opening and said rotating speed is adapted to operatively form said imprinted gel ribbon having a thickness substantially in the order of about 0.035 inches before any die rolling.

9. A rotary die apparatus for forming imprinted gel capsules comprising:
a) a spreader box adapted to hold a flowable gel mass, with said spreader box defining a spreader box opening that is adapted to enable operative flow of the gel mass therethrough;
b) a rotatable casting drum having an exterior cast surface, with said cast surface being provided with surface configurations in embossed relation thereon, and with a receiving portion of said cast surface being in substantial registration with said spreader box opening so as to be adapted to directly receive a dispensed portion of the gel mass therefrom; wherein said casting drum operatively rotates with at least one rotating speed and is adapted to operatively drag the dispensed portion of the gel mass from said spreader box opening to solidify and form said imprinted gel ribbon, with said surface configurations on said cast surface of said casting drum being adapted to form, in relief, an imprinted design in one side of said imprinted gel ribbon;
c) transport means for providing a pair of said imprinted gel ribbons towards a pair of rotary dies; and
d) dispensing means for dispensing a fill material to an injection wedge located between said rotary dies.

10. A rotary die apparatus according to claim 9, further comprising oil rolling means for covering said imprinted gel ribbons with an oil layer before said imprinted gel ribbons are provided to said rotary dies.

11. A rotary die apparatus according to claim 9, wherein said dispensing means comprises a fill pump that is adapted to dispense said fill material to said injection wedge.

12. A rotary die apparatus according to claim 11, wherein said fill pump that is adapted to dispense said fill material under pressure.

13. A rotary die apparatus according to claim 9, wherein said pair of rotary dies are together adapted to form a single web of imprinted gel material from said pair of said imprinted gel ribbons, such that said imprinted design is adapted to be formed in relief in both sides of said web; and wherein said injection wedge and said rotary dies are together adapted to dispense said fill material into encapsulated pocket portions defined within said web.

14. A rotary die apparatus according to claim 13, further comprising chute means for operatively receiving said imprinted gel capsules, after said encapsulated pocket portions are separated from said web.

15. A method for producing an imprinted gel ribbon useful for forming imprinted gel capsules comprising:
a) providing a casting drum having an exterior cast surface with surface configurations formed in embossed relation thereon;
b) flowing a dispensed portion of a gel mass contained in a spreader box, through a spreader box opening therein, directly onto a receiving portion of said exterior cast surface of said casting drum; and
c) rotating said casting drum so as to drag and solidify said dispensed portion of said gel mass, such that said imprinted gel ribbon is formed, with said surface configurations on said cast surface of said casting drum forming, in relief, an imprinted design in one side of said imprinted gel ribbon.

16. A method according to claim 15, wherein in step (c), said cast surface of said casting drum is positively cooled.

17. A method according to claim 15, wherein said imprinted gel ribbon is substantially in the order of about 0.035 inches in thickness before any die rolling.

18. A method according to claim 15, wherein said gel mass comprises gelatin.

19. A method according to claim 15, further comprising step (d) of separating said imprinted gel ribbon from said casting drum as a substantially continuous ribbon.

20. A method according to claim 15, wherein the imprinted gel ribbon is used to form an imprinted gel capsule by the rotary die method.

21. A method for producing an imprinted gel capsule comprising:
a) forming a pair of imprinted gel ribbons by
i) providing a casting drum having an exterior cast surface with surface configurations in embossed relation thereon;
ii) flowing a dispensed portion of a gel mass contained in a spreader box, through a spreader box opening therein, directly onto a receiving portion of said exterior cast surface of said casting drum; and
iii) rotating said casting drum so as to drag and solidify said dispensed portion of said gel mass, such that each of said imprinted gel ribbons is formed, with said surface configurations on said cast surface of said casting drum forming, in relief, an imprinted design in one side of each of said imprinted gel ribbons;
b) feeding the ribbons between a pair of rotary dies containing matching die pockets, whereby a single web of imprinted gel material is formed having encapsulated pocket portions defined therewithin; and
c) filling the encapsulated pocket portions with a fill material.

22. A method according to claim 21, further comprising step (a.1), before step (b), of covering said imprinted gel ribbons with an oil layer.

23. A method according to claim 21, wherein, in step (b), said imprinted design is formed, in relief, in both sides of said single web of imprinted gel material.

24. A method according to claim 21, wherein an encapsulating wall of said web, adjacent to said encapsulated pocket portions, is substantially in the order of about 0.008 inches in thickness.

25. A method according to claim 21, further comprising step (d) of separating said encapsulated pocket portions from said web of imprinted gel material so as to form said imprinted gel capsules.

26. A method according to claim 21, wherein said fill material comprises at least one fill material selected from the group consisting of paintball fill materials, bath bead fill materials, pharmaceutical fill materials, and nutraceutical fill materials.

27. An imprinted and filled gel capsule comprising:
a) a capsule portion formed from a solidified gel material, with said capsule portion having an exterior surface that is provided, in embossed relation, with surface configurations that form, in relief, an imprinted design over substantially the entire extent of said exterior surface of said capsule portion, and with said capsule portion defining an interior encapsulated pocket therewithin; and
b) a fill material contained within said encapsulated pocket of said capsule portion.

28. A gel capsule according to claim 27, wherein said surface configurations are shaped to form at least one imprinted design selected from the group consisting of an embossed pattern, an embossed texture, and an embossed logo.

29. A gel capsule according to claim 28, wherein said surface configurations are shaped to form an embossed dimpled texture.

30. A gel capsule according to claim 27, wherein said capsule portion has a capsule wall that is substantially in the order of about 0.008 inches in thickness.

31. A gel capsule according to claim 27, wherein said gel material comprises gelatin.

32. A gel capsule according to claim 31, wherein said gel material further comprises at least one constituent selected from the group consisting of glycerin, sorbitol, water, titanium, and one or more dyes.

33. A gel capsule according to claim 27, wherein said gel material comprises a synthetic gel material.

34. A gel capsule according to claim 33, wherein said synthetic gel material comprises at least one synthetic gel constituent selected from the group consisting of starches and other polymers.

35. A gel capsule according to claim 27, wherein said fill material comprises at least one fill material selected from the group consisting of paintball fill materials, bath bead fill materials, pharmaceutical fill materials, and nutraceutical fill materials.
